(19) United States
(12) Patent Application Publication
(10) Pub. No.: US 2021/0287588 A1
(43) Pub. Date: Sep. 16, 2021

(54) DISPLAY SYSTEM, DISPLAY CONTROL METHOD, AND ENDOSCOPE SYSTEM

(71) Applicant: OLYMPUS CORPORATION, Tokyo (JP)

(72) Inventor: Yusuke TAJIRI, Tokyo (JP)

(73) Assignee: OLYMPUS CORPORATION, Tokyo (JP)

(21) Appl. No.: 17/331,845

(22) Filed: May 27, 2021

Related U.S. Application Data
(63) Continuation of application No. PCT/JP2018/044269, filed on Nov. 30, 2018.

(51) Int. Cl. G09G 3/20; G09G 5/10; A61B 1/00

(52) U.S. Cl. CPC G09G 3/2003; G09G 2320/046; A61B 1/00045; G09G 5/10

(57) ABSTRACT

A display system includes a memory configured to store comparison information and a processor. The processor receives an image for display including an endoscopic image obtained by picking up an image with an endoscope, generates a display image used for display, outputs the display image to a display, compares information concerning the endoscopic image included in the inputted image for display and the comparison information stored in the memory, obtains a comparison result, and controls, according to the comparison result, whether or not to operate in a burn-in prevention mode.

### CROSS REFERENCE TO RELATED APPLICATION

This application is a continuation application of PCT/JP2018/044269 filed on Nov. 30, 2018, the entire contents of which are incorporated herein by this reference.

### BACKGROUND OF THE INVENTION

### 1. Field of the Invention

The present invention relates to a display system, a display control method, and an endoscope system including a burn-in prevention mode.

### 2. Description of the Related Art

In recent years, an endoscope system has been used in various fields such as a medical field and an industrial field. In the medical field, the endoscope system is used for, for example, observation of an organ in a body cavity, therapeutic treatment performed using a treatment instrument, and a surgical operation under an endoscopic observation. In the endoscope system, an electronic endoscope configured to be capable of picking up a picked-up image in a patient body cavity with an image pickup device is often used. The endoscope system includes an image processing apparatus that performs video processing of a picked-up image (an image pickup signal) obtained by picking up an image with the electronic endoscope. The image processing apparatus can convert the image pickup signal into a video signal of an observation image used for display and the like and output the video signal to a monitor or record the video signal.

However, when the same pattern continues to be displayed on the monitor for a long time, burn-in corresponding to the displayed pattern occurs. In particular, an endoscopic image is displayed in a predetermined region (hereinafter referred to as endoscopic image display region) of the monitor. Burn-in easily occurs in a boundary of the endoscopic image display region because contrast is high in the boundary. Moreover, in the endoscope system, since the endoscopic image display region changes according to a scope in use, visibility of the endoscopic image is markedly deteriorated by burn-in that occurs in a different endoscopic image display region.

In order to prevent such burn-in, a method is conceivable in which a user turns off a power supply of the monitor when display is unnecessary. However, this countermeasure is time-consuming. Therefore, Japanese Patent Application Laid-Open Publication No. 2014-202855 proposes a technique for, when determining that there is no change in temporally adjacent first and second display data displayed on a display unit, displaying, between the first display data and the second display data, burn-in prevention data having a pattern different from the display data. Japanese Patent Application Laid-Open Publication No. H3-231796 proposes a technique for, when a still image is displayed for a predetermined time, automatically changing a display state or urging a user to change the display state.

Note that, on a screen of the monitor of the endoscope system, not only the endoscopic image display region for displaying the endoscopic image but also a status display region for displaying various kinds of status information such as patient information and time information is provided. The display in the status display region sometimes changes even when the endoscopic image does not change.

Operation is sometimes not performed for a long time at an endoscopic test time. However, even in this case, it is sometimes necessary to continue the display of the endoscopic image during the test.

As explained above, even if insertion operation of an endoscope is performed at the endoscopic test time, various kinds of operation for controlling the display of the monitor is sometimes not performed for a long time.

### SUMMARY OF THE INVENTION

A display system according to an aspect of the present invention includes: a memory configured to store comparison information; and a processor. The processor receives an image for display including an endoscopic image obtained by picking up an image with an endoscope, generates a display image used for display, outputs the display image to a display, compares information concerning the endoscopic image included in the inputted image for display and the comparison information stored in the memory, obtains a comparison result, and controls, according to the comparison result, whether or not to operate in a burn-in prevention mode.

An endoscope system according to an aspect of the present invention includes a display system including a display, a memory configured to store comparison information, and a processor. The processor receives an image for display including an endoscopic image obtained by picking up an image with an endoscope, generates a display image used for display, outputs the display image to a display, compares information concerning the endoscopic image included in the inputted image for display and the comparison information stored in the memory, obtains a comparison result, and controls, according to the comparison result, whether or not to operate in a burn-in prevention mode.

A display control method according to an aspect of the present invention includes: receiving an image for display including an endoscopic image obtained by picking up an image with an endoscope and generating a display image used for display; comparing information concerning the endoscopic image included in the inputted image for display and comparison information by using information concerning the image for display before a predetermined period as the comparison information, and outputting a comparison result; and updating the information concerning the image for display when the comparison result indicating that a predetermined condition is not satisfied in the comparison of the information concerning the endoscopic image and the comparison information is outputted and, when the information concerning the endoscopic image and the comparison information continuously coincide a predetermined number of times, regarding that the predetermined condition is satisfied and shifting to a burn-in prevention mode.

### BRIEF DESCRIPTION OF THE DRAWINGS

FIG. 1 is a diagram showing a configuration of a main part of an endoscope system including a display apparatus according to a first embodiment;

FIG. 2 is a flowchart for explaining operation in the first embodiment;

FIG. 3 is a flowchart showing an operation flow adopted in a modification;

FIG. 4 is a block diagram showing a second embodiment of the present invention;

FIG. 5A is an explanatory diagram showing an example of an image displayed on a display screen 33a of a display unit 33;

FIG. 5B is an explanatory diagram showing an example of an image displayed on the display screen 33a of the display unit 33;

FIG. 6A is an explanatory diagram showing an image portion of an endoscopic image display region of an image for display corresponding to FIG. 5A;

FIG. 6B is an explanatory diagram showing an image portion of an endoscopic image display region of an image for display corresponding to FIG. 5B;

FIG. 7A is an explanatory diagram showing RGB values of respective pixels of an endoscopic image Ria1 in the endoscopic image display region shown in FIG. 6A;

FIG. 7B is an explanatory diagram showing RGB values of respective pixels of an endoscopic image Ria2 in the endoscopic image display region shown in FIG. 6B;

FIG. 8 is an explanatory diagram for explaining switching of display;

FIG. 9 is a block diagram showing a third embodiment of the present invention;

FIG. 10 is an explanatory diagram showing an image portion of an endoscopic image display region of a continuously inputted image for display;

FIG. 11 is an explanatory diagram showing an image portion of an endoscopic image display region of a continuously inputted image for display;

FIG. 12 is a block diagram showing a fourth embodiment of the present invention;

FIG. 13 is a block diagram showing a fifth embodiment of the present invention;

FIG. 14 is an explanatory diagram showing an image portion of an endoscopic image display region of a continuously inputted image for display;

FIG. 15 is an explanatory diagram showing an image portion of an endoscopic image display region of a continuously inputted image for display;

FIG. 16 is an explanatory diagram showing an edge pattern extracted from the endoscopic image display region corresponding to FIG. 14;

FIG. 17 is an explanatory diagram showing an edge pattern extracted from the endoscopic image display region corresponding to FIG. 15; and

FIG. 18 is a flowchart showing the fifth embodiment of the present invention.

### DETAILED DESCRIPTION OF THE PREFERRED EMBODIMENTS

Embodiments of the present invention are explained in detail below with reference to the drawings.

### First Embodiment

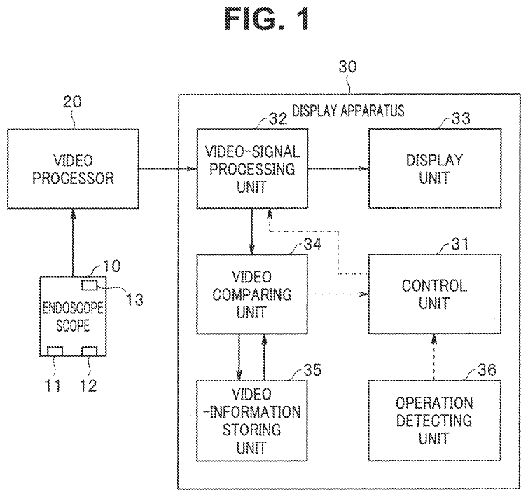

FIG. 1 is a diagram showing a configuration of a main part of an endoscope system including a display apparatus according to a first embodiment. In the present embodiment, comparison is made, about an image portion of an endoscopic image display region in an image displayed on the display apparatus, on images under a predetermined comparison condition and, when a comparison result satisfies a predetermined condition, the mode is shifted to a burn-in prevention mode. Consequently, even at a suspension time and the like of an endoscopic test, it is possible to surely shift to the burn-in prevention mode, and to prevent burn-in of the display apparatus.

As shown in FIG. 1, an endoscope system includes an endoscope scope 10, a video processor 20, and a display apparatus 30.

The endoscope scope 10 includes a not-shown insertion section having an elongated shape insertable into a body cavity of a subject. An image pickup unit 11 is provided at a distal end portion of the insertion section. A not-shown light source apparatus 12 is provided in the insertion section. The light source apparatus 12 is configured to be able to irradiate illumination light on an object. The image pickup unit 11 includes an image sensor such as a color CCD or color CMOS sensor and is configured such that light reflected from the object forms an image in a light receiving unit. The image pickup unit 11 photoelectrically converts an optical image reflected from the object to acquire an image pickup signal and outputs the acquired image pickup signal to the video processor 20. A memory 13 is provided in the endoscope scope 10. A scope information indicating a scope type is stored in the memory 13.

The video processor 20 controls driving of the image pickup unit 11 and the light source apparatus 12 and receives the image pickup signal from the image pickup unit 11. The video processor 20 is configured to read out the scope information from the memory 13. The video processor 20 applies predetermined signal processing to the received image pickup signal to thereby generate an endoscopic image of the object and applies highlighting processing and white balance correction processing to the generated endoscopic image. Further, the video processor 20 generates a video signal of an image for display including a status information display region for displaying status information such as patient information and time information and an endoscopic image display region for displaying the endoscopic image. The video processor 20 outputs the generated image for display (the video signal) to the display apparatus 30. The video processor 20 is configured to output the scope information to the display apparatus 30 as well.

The display apparatus 30 functioning as a display system is configured by a control unit 31, a video-signal processing unit 32, a display unit 33, a video comparing unit 34, a video-information storing unit 35, and an operation detecting unit 36. Note that, as the display unit 33, a display panel such as a liquid crystal panel and various displays such as a display that performs two-dimensional display or three-dimensional display such as a projector and a display having a flexible display surface can be adopted. The control unit 31, the video-signal processing unit 32, the video comparing unit 34, and the operation detecting unit 36 may be configured by a processor in which a CPU, an FPGA, or the like is used or may operate according to a program stored in a not-shown memory. A part or all of functions of the control unit 31, the video-signal processing unit 32, the video comparing unit 34, and the operation detecting unit 36 may be realized by a hardware electronic circuit. The control unit 31 controls the units in the display apparatus 30.

Note that, in the following explanation, an example is explained in which the display apparatus 30 displays a display image on the display unit 33. However, the display apparatus 30 may function as a display control circuit that outputs the display image to a display configured by a display panel or the like provided on an outside.

The video-signal processing unit 32 outputs, to the display unit 33, a display image generated by applying predetermined video signal processing to the video signal (the image for display) sent from the video processor 20. When the burn-in prevention mode is designated by the control unit 31, the video-signal processing unit 32 applies predetermined burn-in prevention processing to the image for display. For example, as the burn-in prevention mode, the video-signal processing unit 32 may generate a display image less easily burning in different from the inputted image for display and output the display image to the display unit 33, may output a display image in a predetermined white level, or may stop the output of the display image to the display unit 33.

The video signal (the image for display) to which the burn-in prevention processing has not been applied is inputted from the video-signal processing unit 32 to the video comparing unit 34 functioning as a comparing unit. The video comparing unit 34 outputs the inputted video signal (image for display) to the video-information storing unit 35. The video-information storing unit 35 functioning as an information storing unit is configured to store comparison information used for comparison processing of the video comparing unit 34. For example, the video-information storing unit 35 stores, as the comparison information, the image for display sent from the video comparing unit 34. The video-information storing unit 35 retains the stored image for display for a predetermined period and outputs the stored image to the video comparing unit 34 and updates, with the image for display inputted from the video comparing unit 34, an image for display stored before the image for display. The video comparing unit 34 performs the update of the comparison information (the image for display) stored in the video-information storing unit 35 when a predetermined comparison condition explained below is not satisfied. Note that the video comparing unit 34 may sometimes perform the update of the comparison information of the video-information storing unit 35 every time an image for display is inputted to the video comparing unit 34.

The video comparing unit 34 compares the comparison information (the image for display) stored in the video-information storing unit 35 and the inputted image for display. Consequently, for example, the video comparing unit 34 performs comparison of images for display before and after the predetermined period. In the present embodiment, the video comparing unit 34 is configured to perform the comparison about an endoscopic image display region.

The video comparing unit 34 carries out this comparison based on a predetermined comparison condition and determines whether or not a comparison result satisfies a predetermined condition. When the comparison result satisfies the predetermined condition, the video comparing unit 34 transmits, to the control unit 31, a first instruction signal indicating that the mode may be shifted to the burn-in prevention mode. For example, the video comparing unit 34 may include a processing unit for image analysis processing and count processing and may be configured to perform comparison of coincidence and non-coincidence of two images before and after the predetermined period about the endoscopic image display region. When coincidence determination reaches a predetermined number of times, the video comparing unit 34 may be configured to regard that the predetermined condition is satisfied, and transmit a first instruction signal indicating that the mode may be shifted to the burn-in prevention mode.

Usually, the video processor 20 sets a different endoscopic image display region for each scope type of the endoscope scope 10. Therefore, the video-information storing unit 35 is configured to store a table describing a correspondence relation between scope types and positions (regions) in images in endoscopic image display regions in the case in which scopes of the scope types are used.

The video comparing unit 34 is configured to receive scope information from the video-signal processing unit 32 as well. The video comparing unit 34 is configured to refer to the table of the video-information storing unit 35 using the scope information acquired from the video-signal processing unit 32 to grasp an endoscopic image display region corresponding to an endoscope scope currently in use and perform comparison of images about the region.

In the display apparatus 30, various operation sections such as not-shown switches, buttons, and dials are disposed. The operation detecting unit 36 is configured to detect presence or absence of operation on these operation sections in the display apparatus 30 and calculate a continuation period of a non-operation state. When detecting that the non-operation state on the operation sections continues for a predetermined time, the operation detecting unit 36 transmits a second instruction signal indicating that the mode may be shifted to the burn-in prevention mode.

The control unit 31 is configured to, when receiving, from the video comparing unit 34, the first instruction signal indicating that the mode may be shifted to the burn-in prevention mode and receiving, from the operation detecting unit 36, the second instruction signal indicating that the mode may be shifted to the burn-in prevention mode, transmit a command for shifting to the burn-in prevention mode to the video-signal processing unit 32.

Note that the control unit 31 may be configured to, irrespective of the second instruction signal from the operation detecting unit 36, when receiving the first instruction signal from the video comparing unit 34, transmit the command for shifting to the burn-in prevention mode to the video-signal processing unit 32.

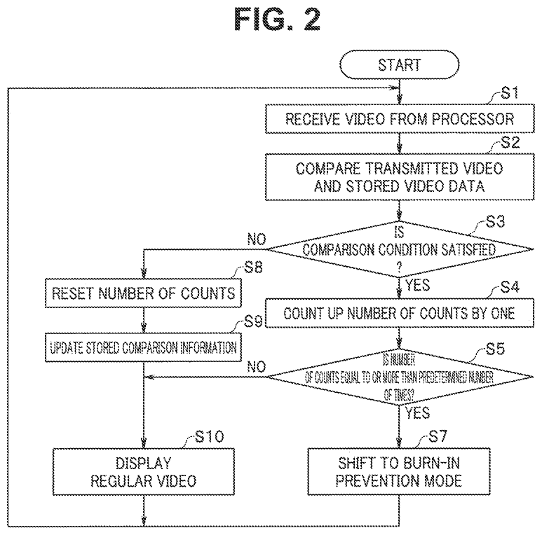

Subsequently, operation in the embodiment configured as explained above is explained with reference to FIG. 2. FIG. 2 is a flowchart for explaining operation in the first embodiment.

The endoscope scope 10 is controlled by the video processor 20 and picks up an image of an object and outputs an image pickup signal to the video processor 20. The video processor 20 receives the image pickup signal from the endoscope scope 10 and acquires the scope information stored in the memory 13 of the endoscope scope 10. The video processor 20 applies predetermined signal processing to the image pickup signal, generates a video signal for an image for display, and outputs the video signal to the display apparatus 30. The video processor 20 outputs the scope information to the display apparatus 30 as well. The scope information is received by the video-signal processing unit 32 of the display apparatus 30 and supplied to the video comparing unit 34. The video comparing unit 34 refers to, based on the scope information, the table stored in the video-information storing unit 35 and acquires information concerning an endoscopic image display region corresponding to the endoscope scope 10.

In step S1 in FIG. 2, the video-signal processing unit 32 of the display apparatus 30 receives an image for display from the video processor 20. The video-signal processing unit 32 generates a display image based on the received image for display and sequentially outputs the display image to the display unit 33. The video-signal processing unit 32 sequentially outputs the received image for display to the video comparing unit 34. The display unit 33 performs display based on the display image, that is, displays, on a display screen, an image including status information and an endoscopic image display region.

The video-comparing unit 34 outputs the inputted image for display to the video-information storing unit 35 and causes the video-information storing unit 35 to store the image for display, reads out, from the video-information storing unit 35, an image for display before the predetermined period, and performs comparison about an endoscopic image display region between a currently inputted image for display and an image for display in the past.

The video comparing unit 34 determines whether or not the compared images satisfy a predetermined comparison condition. For example, the video comparing unit 34 adopts a coincidence condition, a condition of comparison of which is whether or not the images in the endoscopic image display region coincide.

It is assumed that an endoscopic test is suspended. Even in this case, power supplies of the endoscope scope 10, the video processor 20, and the display apparatus 30 are sometimes in an ON state. However, in this case, a change does not occur in the images in the endoscopic image display region. Therefore, in such a situation, the video comparing unit 34 determines that the coincidence condition is satisfied.

The video comparing unit 34 further determines a number of times condition about whether or not the comparison condition is continuously satisfied a predetermined number of times. For this determination, the video comparing unit 34 is configured to, when determining in step S3 that the comparison condition (for example, the coincidence condition) is satisfied, count up the number of counts by one in step S4. In the next step S5, the video comparing unit 34 determines whether or not the number of counts has reached the predetermined number of times. When the number of counts has not reached the predetermined number of times, in step S10, the display unit 33 continues the display of the present display image.

After the processing in step S10, the processing in step S1 and subsequent steps is repeated. When determining in step S3 that two images about the endoscopic image display region do not satisfy the predetermined comparison condition, the video comparing unit 34 shifts the processing to step S8 and resets the number of counts. In the next step S9, the video comparing unit 34 updates the comparison information (the image for display) of the video-information storing unit 35 and proceeds to step S10.

When determining that the number of times the two images about the endoscopic image display region satisfy the predetermined comparison condition is equal to or more than the predetermined number of times, the video comparing unit 34 outputs, to the control unit 31, a first instruction signal indicating that the mode may be shifted to the burn-in prevention mode. Consequently, the control unit 31 instructs the video-signal processing unit 32 to shift to the burn-in prevention mode (step S7).

The video-signal processing unit 32 prevents burn-in of the display unit 33 by, for example, stopping an output of a display image to the display unit 33 or outputting a display image in a predetermined white level to the display unit 33.

Note that, in FIG. 2, an example in which the condition for the shift to the burn-in prevention mode is to satisfy the comparison condition and the number of times condition is explained. However, the mode may be shifted to the burn-in prevention mode when the comparison condition is satisfied once.

In this way, in the present embodiment, the endoscope system shifts to the burn-in prevention mode by comparing, under a predetermined comparison condition, images before and after the predetermined period about an image portion of an endoscopic image display region in an image displayed on the display apparatus. Consequently, even at a suspension time and the like of an endoscopic test, it is possible to shift to the burn-in prevention mode, and to surely prevent burn-in of the display apparatus.

### Modification

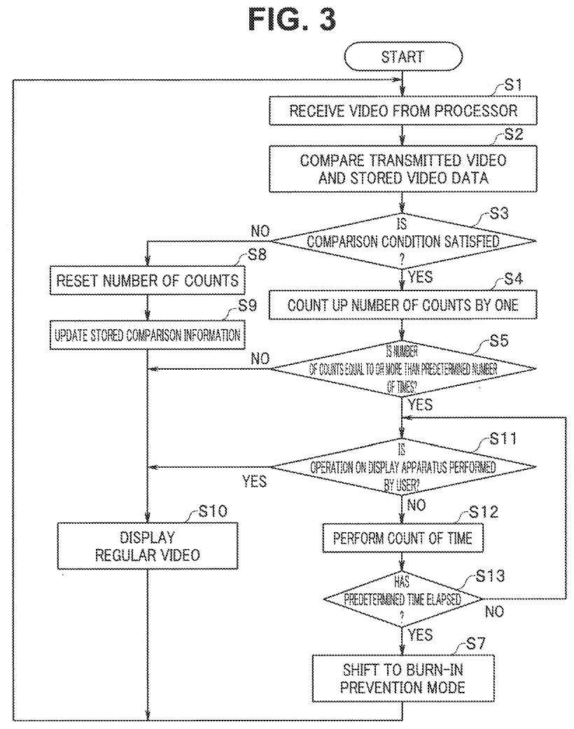

FIG. 3 is a flowchart showing an operation flow adopted in a modification. This modification is an example in which the endoscope system shifts to the burn-in prevention mode according to the first instruction signal and the second instruction signal.

FIG. 3 is different from FIG. 2 in that steps S11 to S13 are added. When the number of times the comparison condition is satisfied is equal to or more than the predetermined number of times, the processing shifts from step S5 to step S11. The operation detecting unit 36 determines whether or not a user is performing operation on the display apparatus 30. When operation is not performed, the operation detecting unit 36 starts counting a time in step S12 and determines in step S13 whether or not a predetermined time has elapsed. When the predetermined time has not elapsed, the operation detecting unit 36 returns the processing to step S11 and determines presence or absence of operation on the display apparatus 30. When determining in steps S11 to S13 that operation on the display apparatus 30 is not performed for the predetermined time, the operation detecting unit 36 outputs, to the control unit 31, the second instruction signal indicating the mode may be shifted to the burn-in prevention mode. Note that when the operation on the display apparatus 30 does not continue for the predetermined time, the processing shifts from step S11 to step S10 and a display image based on the image for display is displayed on the display unit 33.

The other action is the same as the action in the first embodiment.

Note that, in FIG. 3, after the video comparing unit 34 determines that the endoscope system should shift to the burn-in prevention mode, the operation detecting unit 36 determines presence or absence of operation. However, the determination of the video comparing unit 34 and the operation detecting unit 36 may be independently and simultaneously performed.

### Second Embodiment

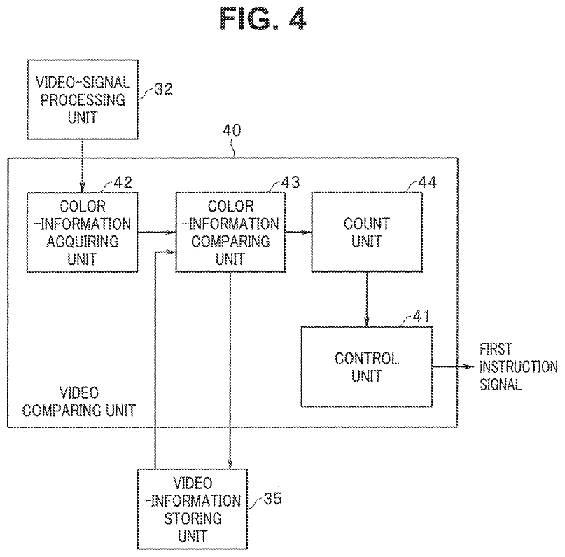

FIG. 4 is a block diagram showing a second embodiment of the present invention. An endoscope system in the present embodiment is different from the one shown in FIG. 1 in that a video comparing unit 40 is adopted instead of the video comparing unit 34 in the display apparatus 30. The other components are the same as the components in the first embodiment. Explanation about the components is omitted. FIG. 4 shows an example of a specific configuration of the video comparing unit 40. The present embodiment shows an example in which coincidence of color information of videos is a comparison condition.

The video comparing unit 40 is configured by a control unit 41, a color-information acquiring unit 42, a color-information comparing unit 43, and a count unit 44. The control unit 41 and the color-information comparing unit 43 may be configured by a processor in which a CPU, an FPGA, or the like is used or may operate according to a program stored in a not-shown memory, or a part or all of functions of the control unit 41 and the color-information comparing unit 43 may be realized by a hardware electronic circuit. The control unit 41 controls the units of the video comparing unit 40.

The color-information acquiring unit 42 acquires, about an image for display supplied from the video-signal processing unit 32, pixel by pixel, color information (for example, RGB values) of all pixels in an endoscopic image display region. The color-information acquiring unit 42 outputs the acquired RGB values in the endoscopic image display region to the color-information comparing unit 43.

The color-information comparing unit 43 reads out, from the video-information storing unit 35, RGB values, which are comparison information, stored before a predetermined period and compares the RGB values with present RGB values received from the color-information acquiring unit 42. When a comparison condition explained below concerning color information is not satisfied, the color-information comparing unit 43 gives the inputted RGB values to the video-information storing unit 35 and causes the video-information storing unit 35 to update storage. The color-information comparing unit 43 determines whether or not RGB values in respective pixel positions in endoscopic image display regions of images for display before and after the predetermined period coincide. When the RGB values in the respective pixel positions in the endoscopic image display regions coincide about all the pixels, the color-information comparing unit 43 determines that the comparison condition is satisfied. Note that even when pixels equal to or smaller than a predetermined number do not coincide, the color-information comparing unit 43 may determine that the comparison condition is satisfied.

The color-information comparing unit 43 compares the RGB values in every predetermined period, for example, every frame, and outputs to the count unit 44 a comparison result about whether or not the comparison condition is satisfied. The count unit 44 counts the comparison result from the color-information comparing unit 43. The count unit 44 counts up a count value every time a comparison result indicating that the comparison condition is satisfied is given and initializes the count value when a comparison result indicating that the comparison condition is not satisfied is given. The count unit 44 outputs the count value to the control unit 41.

The control unit 41 determines whether or not the count value has reached a predetermined value. The count unit 44 is configured to, when the count value has reached the predetermined value, that is, when the color-information comparing unit 43 outputs a comparison result indicating that the comparison condition is continuously satisfied a predetermined number of times, generate a first instruction signal indicating that the mode may be shifted to a burn-in prevention mode and output the first instruction signal to the control unit 31.

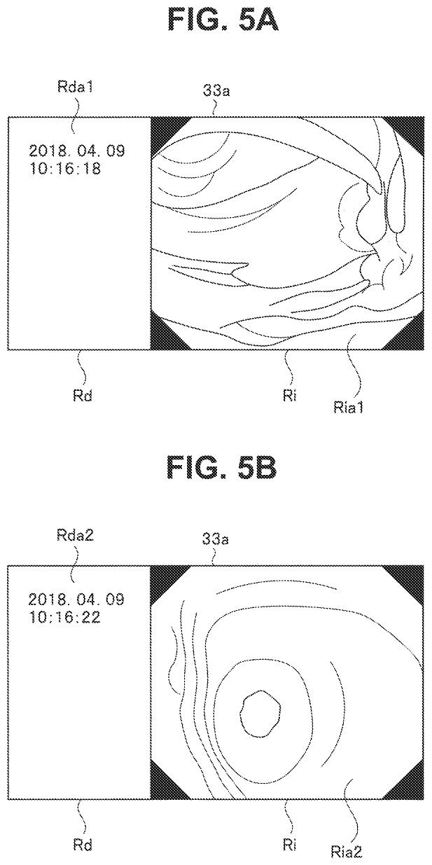

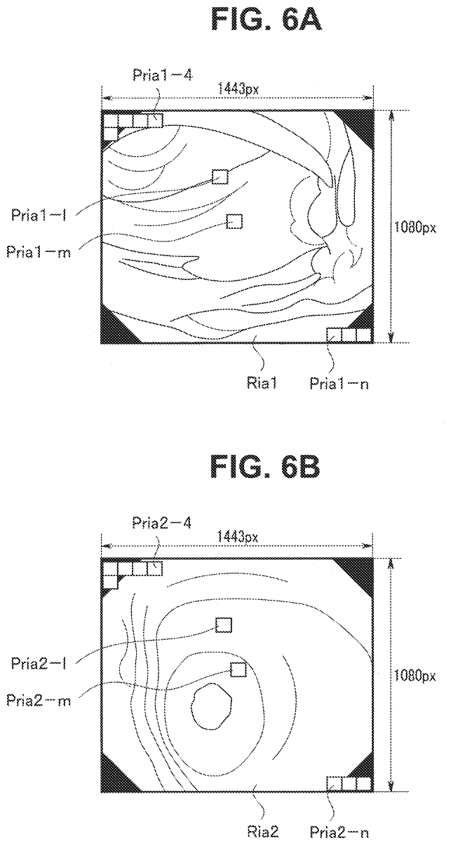

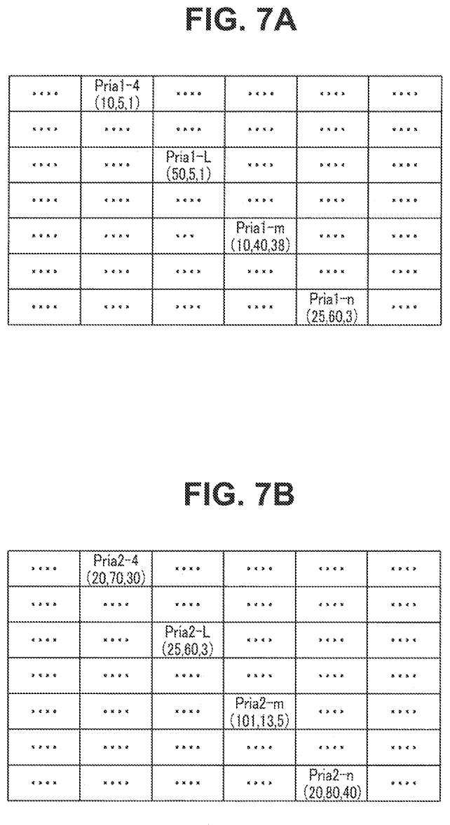

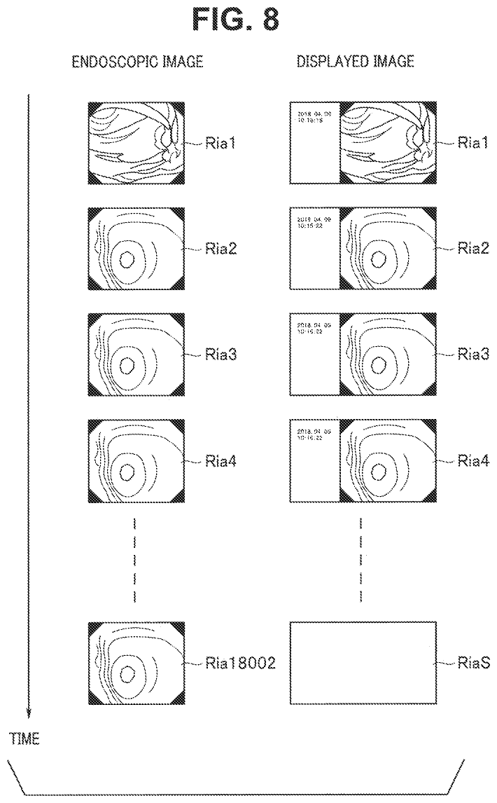

Subsequently, operation in the embodiment configured as explained above is explained with reference to explanatory diagrams of FIG. 5A, FIG. 5B, FIG. 6A, FIG. 6B, FIG. 7A, FIG. 7B, and FIG. 8. FIG. 5A and FIG. 5B show examples of images displayed on the display screen 33a of the display unit 33. FIG. 6A and FIG. 6B show image portions in endoscopic image display regions of images for display respectively corresponding to FIG. 5A and FIG. 5B.

It is assumed that display images of before and after predetermined one frame are the images shown in FIG. 5A and FIG. 5B. The display image before one frame shown in FIG. 5A includes an endoscopic image display region Ri and a status information display region Rd. An endoscopic image Ria1 is displayed in the endoscopic image display region Ri. A status information display Rda1 is displayed in the status information display region Rd.

FIG. 5B shows a present display image after one frame shown in FIG. 5A. The present display image shown in FIG. 5B also includes the endoscopic image display region Ri and the status information display region Rd. An endoscopic image Ria2 is displayed in the endoscopic image display region Ri. A status information display Rda2 is displayed in the status information display region Rd.

The color-information acquiring unit 42 receives an image for display from the video-signal processing unit 32 and acquires RGB values in an endoscopic image display region of the inputted image for display. Square marks in FIG. 6A and FIG. 6B indicate a part of pixels in the endoscopic image display region. For example, the endoscopic image display region is formed by horizontal 1443 pixels (px)×vertical 1080 pixels (px). Respective pixel positions in the endoscopic image display region of pixels Pria1-4, Pria1-1, Pria1-m, and Pria1-n in FIG. 6A and respective pixel positions in the endoscopic image display region of pixels Pria2-4, Pria2-1, Pria2-m, and Pria2-n in FIG. 6B are the same.

FIG. 7A shows RGB values of respective pixels of an endoscopic image Ria1 in the endoscopic image display region shown in FIG. 6A. FIG. 7B shows RGB values of respective pixels of an endoscopic image Ria2 in the endoscopic image display region shown in FIG. 6B. In FIG. 7A and FIG. 7B, “ . . . ” indicates one or a plurality of rows and columns. In an example shown in FIG. 7A, RGB pixel values of the pixels Pria1-4, Pria1-1, Pria1-m, and Pria1-n in the endoscopic image Ria1 are respectively (10, 5, 1), (50, 5, 1), (10, 40, 38), and (25, 60, 3). In an example shown in FIG. 7B, RGB pixel values of the pixels Pria2-4, Pria2-1, Pria2-m, and Pria2-n in the endoscopic image Ria2 are respectively (20, 70, 30), (25, 60, 3), (101, 13, 5), and (20, 80, 40).

It is assumed that, at timing when the image for display corresponding to FIG. 5B is inputted to the color-information acquiring unit 42 from the video-signal processing unit 32, the RGB values shown in FIG. 7A based on the endoscopic image Ria1 shown in FIG. 5A are stored in the video-information storing unit 35. The color-information acquiring unit 42 acquires the RGB values shown in FIG. 7B and outputs the RGB values to the color-information comparing unit 43.

The color-information comparing unit 43 compares, for each of pixels in the same position, the RGB values shown in FIG. 7A and the RGB values shown in FIG. 7B and determines whether or not RGB values coincide in all the pixel positions. In the examples shown in FIG. 7A and FIG. 7B, it is determined that the RGB values do not coincide.

FIG. 8 is a diagram for explaining switching of display. As shown in FIG. 8, it is assumed that endoscopic images Ria1, Ria2, Ria3, . . . , Ria18001, Ria18002, . . . in an endoscopic image display region Ri are sequentially inputted to the color-information acquiring unit 42 for each of frames. In the endoscopic image Ria1 and the endoscopic image Ria2, RGB values of all pixels in the endoscopic image display region Ri do not coincide. In the endoscopic images Ria3, Ria4, . . . inputted after the endoscopic image Ria2, it is assumed that RGB values in respective pixel positions in the endoscopic image display region Ri coincide about all pixels. Therefore, after a point in time when the RGB values based on the endoscopic image Ria3 are inputted to the color-information comparing unit 43, a comparison result indicating that the comparison condition is satisfied is outputted to the count unit 44 from the color-information comparing unit 43. The count unit 44 is initialized by a comparison result indicating that the comparison condition is not satisfied and counts up a count value every time the comparison result indicating that the comparison condition is satisfied is given and outputs the count value to the control unit 41.

If it is assumed that a frame frequency is 60 fps (frames/second) and the first instruction signal indicating that the mode may be shifted to the burn-in prevention mode is generated when the comparison condition is continuously satisfied for five minutes, the control unit 41 determines whether or not the count value has reached (60×300)=18000. In an example shown in FIG. 8, when the color-information comparing unit 43 performs coincidence determination about the endoscopic image Ria2 and the endoscopic image Ria18002, the first instruction signal indicating that the mode may be shifted to the burn-in prevention mode is outputted from the control unit 41.

Until the first instruction signal is generated, the video-signal processing unit 32 generates a display image based on the inputted image for display and outputs the display image to the display unit 33. As shown in FIG. 8, the endoscopic images Ria1, Ria2, . . . , and Ria18001 are sequentially displayed. When the endoscopic image Ria18002 is inputted and the first instruction signal is generated, the video-signal processing unit 32 outputs a display image for burn-in prevention to the display unit 33. In the example shown in FIG. 8, a display image RiaS in a predetermined white level is displayed.

As explained above, in the present embodiment, the RGB values are calculated about the respective pixels in the endoscopic image display region, about the images for display before and after the predetermined period, the RGB values of the pixels in the same position in the endoscopic image display region coinciding about all the pixels in the endoscopic image display region is set as the comparison condition, and the first instruction signal indicating that the mode may be shifted to the burn-in prevention mode is generated when the comparison condition is continuously satisfied for the predetermined period. Consequently, even at a suspension time and the like of an endoscopic test, it is possible to shift to the burn-in prevention mode, and to surely prevent burn-in of the display apparatus.

Note that, in the present embodiment as well, the modification shown in FIG. 3 may be adopted and the burn-in prevention mode may be set based on the first and second instruction signals.

### Third Embodiment

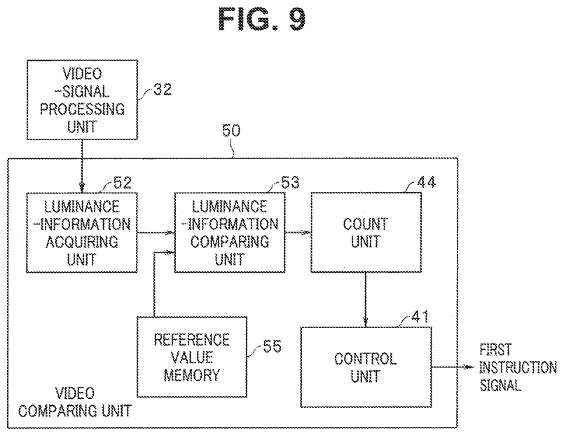

FIG. 9 is a block diagram showing a third embodiment of the present invention. An endoscope system in the present embodiment is different from the one shown in FIG. 1 in that a video comparing unit 50 is adopted instead of the video comparing unit 34 in the display apparatus 30. The other components are the same as the components in the first embodiment. Explanation about the components is omitted. FIG. 9 shows an example of a specific configuration of the video comparing unit 50. The video comparing unit 50 is different from the video comparing unit 40 shown in FIG. 4 in that a luminance-information acquiring unit 52 and a luminance-information comparing unit 53 are adopted respectively instead of the color-information acquiring unit 42 and the color-information comparing unit 43 and a reference value memory 55 is added. The present embodiment shows an example in which the magnitude of the luminance of a video is set as a comparison condition.

The luminance-information comparing unit 53 may be configured by a processor in which a CPU, an FPGA, or the like is used or may operate according to a program stored in a not-shown memory or a part or all of functions of the luminance-information comparing unit 53 may be realized by a hardware electronic circuit. The control unit 41 controls the units in the video comparing unit 50.

The luminance-information acquiring unit 52 acquires, about an image for display supplied from the video-signal processing unit 32, pixel by pixel, luminance values of all pixels in an endoscopic image display region. The luminance-information acquiring unit 52 outputs the acquired luminance values in the endoscopic image display region to the luminance-information comparing unit 53.

The reference value memory 55 functioning as an information storing unit stores, as comparison information, a reference luminance value for determining a shift to a burn-in prevention mode. For example, the reference luminance value is set to a predetermined black level. For example, the reference luminance value may be set to a value slightly higher than an average luminance value of an endoscopic image display region of an image for display outputted from the video-signal processing unit 32 when the light source apparatus 12 is extinguished.

The luminance-information comparing unit 53 calculates an average of the luminance values inputted from the luminance-information acquiring unit 52 (hereinafter referred to as average luminance value). The luminance-information comparing unit 53 compares the calculated average luminance value and the reference luminance value read out from the reference value memory 55. When the average luminance value is equal to or smaller than the reference luminance value, the luminance-information comparing unit 53 outputs, to the count unit 44, a comparison result indicating that the average luminance value decreases to the reference luminance value or less. The count unit 44 performs counting of the comparison result and outputs the number of times the average luminance value decreases to the reference luminance value or less to the control unit 41.

In the present embodiment, the control unit 41 is configured to, when a count value indicating that the comparison result indicating that the average luminance value decreases to the reference luminance value or less is generated once or continuously generated several times is given from the count unit 44, output, to the control unit 31, a first instruction signal indicating that the mode may be shifted to the burn-in prevention mode.

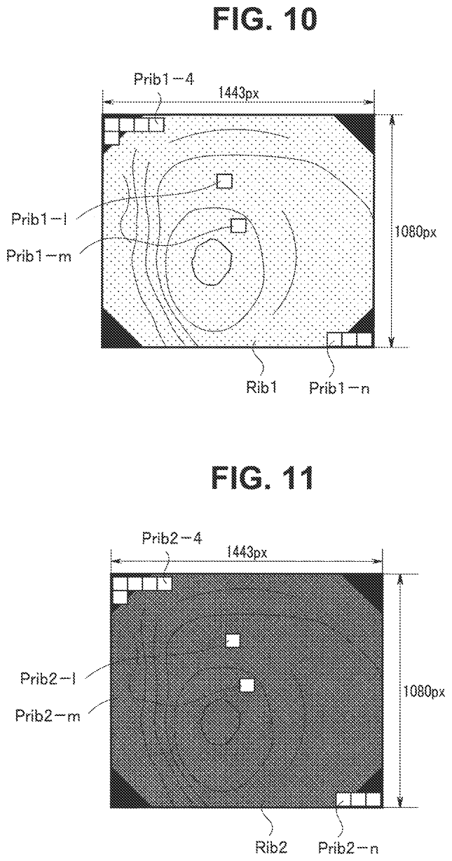

Subsequently, operation in the embodiment configured as explained above is explained with reference to explanatory diagrams of FIG. 10 and FIG. 11. FIG. 10 and FIG. 11 show image portions of endoscopic image display regions of continuously inputted images for display.

It is assumed that display images before and after predetermined one frame are the images shown in FIG. 10 and FIG. 11. An image in an endoscopic image display region in the image for display before one frame shown in FIG. 10 is an endoscopic image Rib1. An image in an endoscopic image display region of a present image for display shown in FIG. 11 after one frame shown in FIG. 10 is an endoscopic image Rib2.

Note that the endoscopic image Rib1 shown in FIG. 10 indicates an image for display obtained at a normal endoscopic test time and indicates, with hatching, that the image for display is a relatively bright image. The endoscopic image Rib2 shown in FIG. 11 indicates an image for display obtained at an extinction time of the light source apparatus 12 and indicates, with hatching, that the image for display is a dark image.

The luminance-information acquiring unit 52 receives an image for display from the video-signal processing unit 32 and acquires a luminance value in an endoscopic image display region of the inputted image for display. Square marks in FIG. 10 and FIG. 11 indicate a part of pixels in the endoscopic image display region. For example, the endoscopic image display region is formed by horizontal 1443 pixels (px)×vertical 1080 pixels (px). Respective pixel positions in the endoscopic image display region of pixels Prib1-4, Prib1-1, Prib1-m, and Prib1-n in FIG. 10 and respective pixel positions in the endoscopic image display region of pixels Prib2-4, Prib2-1, Prib2-m, and Prib2-n in FIG. 11 are the same.

The luminance-information acquiring unit 52 sequentially receives an image for display from the video-signal processing unit 32, acquires luminance values of respective pixels in an endoscopic image display region, and outputs the luminance values to the luminance-information comparing unit 53. The luminance-information comparing unit 53 calculates an average luminance value by adding up the inputted luminance values in the endoscopic image display region and dividing the added-up value by the number of pixels in the endoscopic image display region. The luminance-information comparing unit 53 reads out the reference luminance value from the reference value memory 55 and compares the average luminance value and the reference luminance value. When the average luminance value is equal to or smaller than the reference luminance value, the luminance-information comparing unit 53 outputs a comparison result indicating to that effect to the count unit 44.

The count unit 44 outputs, to the control unit 41, the number of times the average luminance value is continuously equal to or smaller than the reference value. When a count value of the count unit 44 is 1 or when the count value is, for example, 2, the control unit 41 generates the first instruction signal indicating that the mode may be shifted to the burn-in prevention mode and outputs the first instruction signal to the control unit 31.

The other action is the same as the action in the first embodiment.

As explained above, in the present embodiment, the luminance values are calculated about the respective pixels in the endoscopic image display region, whether or not an average luminance value of the endoscopic image display region is equal to or smaller than a predetermined reference luminance value is set as the comparison condition, and the first instruction signal indicating that the mode may be shifted to the burn-in prevention mode is generated when the comparison condition is satisfied once or continuously satisfied several times. Consequently, at a suspension time and the like of an endoscopic test, even if only the light source apparatus is turned off, it is possible to shift to the burn-in prevention mode, and to surely prevent burn-in of the display apparatus.

Note that, in the present embodiment as well, the modification shown in FIG. 3 may be adopted and the burn-in prevention mode may be set based on the first and second instruction signals.

### Fourth Embodiment

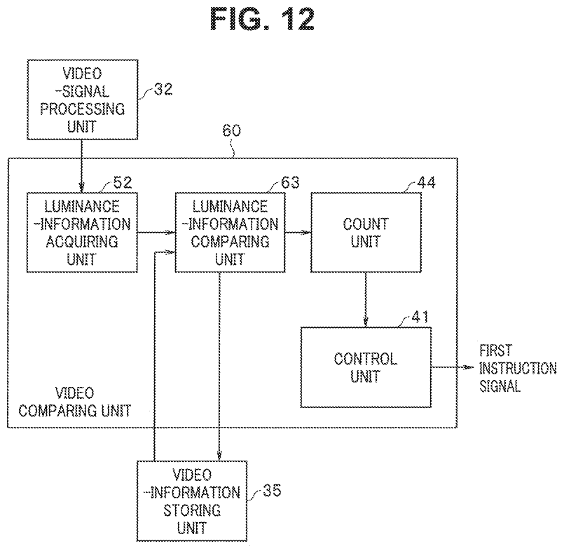

FIG. 12 is a block diagram showing a fourth embodiment of the present invention. An endoscope system in the present embodiment is different from the one shown in FIG. 1 in that a video comparing unit 60 is adopted instead of the video comparing unit 34 in the display apparatus 30. The other components are the same as the components in the first embodiment. Explanation about the components is omitted. FIG. 12 shows an example of a specific configuration of the video comparing unit 60. The video comparing unit 60 is different from the video comparing unit 50 shown in FIG. 9 in that the reference value memory 55 is omitted and a luminance-information comparing unit 63 is adopted instead of the luminance-information comparing unit 53. The present embodiment also shows an example in which the magnitude of the luminance of a video is set as a comparison condition.

The luminance-information comparing unit 63 may be configured by a processor in which a CPU, an FPGA, or the like is used or may operate according to a program stored in a not-shown memory, or a part or all of functions of the luminance-information comparing unit 63 may be realized by a hardware electronic circuit. The control unit 41 controls the units in the video comparing unit 60.

The luminance-information comparing unit 63 calculates an average (an average luminance value) of luminance values inputted from the luminance-information acquiring unit 52. The luminance-information comparing unit 63 reads out, from the video-information storing unit 35, an average luminance value (comparison information) stored before a predetermined period (for example, before one frame) and compares the average luminance value with a present average luminance value calculated from the output of the luminance-information acquiring unit 52. The luminance-information comparing unit 63 determines whether or not an average luminance value in endoscopic image display regions of images for display before and after the predetermined period decreases by a predetermined threshold or more. When the average luminance value in the endoscopic image display regions decreases by a predetermined value or more from an average luminance value stored before the predetermined period, the luminance-information comparing unit 63 determines that the comparison condition is satisfied. When the comparison condition is not satisfied, the luminance-information comparing unit 63 gives the calculated average luminance value to the video-information storing unit 35 and causes the video-information storing unit 35 to update storage.

Note that the predetermined value used for the comparison in the luminance-information comparing unit 63 is set to a value slightly smaller than a difference between average luminance values of endoscopic image display regions of images for display outputted from the video-signal processing unit 32 at a normal endoscopic test time and an extinction time of the light source apparatus 12.

The luminance-information comparing unit 63 performs comparison of average luminance values in every predetermined period, for example, every frame, and outputs to the count unit 44 a comparison result about whether or not the comparison condition is satisfied. The count unit 44 calculates the number of times the comparison condition is continuously satisfied and outputs the number of times to the control unit 41.

In the present embodiment as well, the control unit 41 is configured to, when a count value indicating that the comparison result indicating that the average luminance value decreases to the reference luminance value or less is generated once or continuously generated several times is given from the count unit 44, output, to the control unit 31, a first instruction signal indicating that the mode may be shifted to a burn-in prevention mode.

The embodiment configured as explained above is different from the third embodiment in that the luminance-information comparing unit 63 compares the present average luminance value with the average luminance value stored before the predetermined period. For example, at a suspension time and the like of an endoscopic test, in some case, only the light source apparatus 12 of the endoscope scope 10 is extinguished and power supplies of the video processor 20 and the display apparatus 30 are in an ON state. In this case, immediately after the light source apparatus 12 is turned off, the present average luminance value decreases by a predetermined value or more from an average luminance value before one frame. A comparison result indicating that the comparison condition is satisfied is outputted from the luminance-information comparing unit 63 to the count unit 44.

In this way, immediately after the light source apparatus 12 is turned off, the first instruction signal indicating that the mode may be shifted to the burn-in prevention mode is outputted from the control unit 41 to the control unit 31.

The other action is the same as the action in the third embodiment.

As explained above, in the present embodiment as well, the luminance values are calculated about the respective pixels in the endoscopic image display region, the average luminance value of the endoscopic image display region decreasing by the predetermined value or more from the average luminance value stored before the predetermined period is set as the comparison condition, and the first instruction signal indicating that the mode may be shifted to the burn-in prevention mode is generated when the comparison condition is satisfied once or continuously satisfied several times. Consequently, at a suspension time and the like of an endoscopic test, even if only the light source apparatus is turned off, it is possible to shift to the burn-in prevention mode, and to surely prevent burn-in of the display apparatus.

In the present embodiment, when the average luminance value decreases by the predetermined value or more for the first time, the update of the video-information storing unit 35 may be stopped and an average luminance value before the average luminance value decreases by the predetermined value or more may be stored in the video-information storing unit 35. In this case, for example, the control unit 41 may be configured to, when the number of counts in the case of 60 fps reaches 600 (10 seconds elapse), generate the first instruction signal indicating that the mode may be shifted to the burn-in prevention mode. Then, when a light source apparatus is turned off for ten seconds, it is possible to shift to the burn-in prevention mode.

Note that, in the present embodiment as well, the modification shown in FIG. 3 may be adopted and the burn-in prevention mode may be set based on the first and second instruction signals.

### Fifth Embodiment

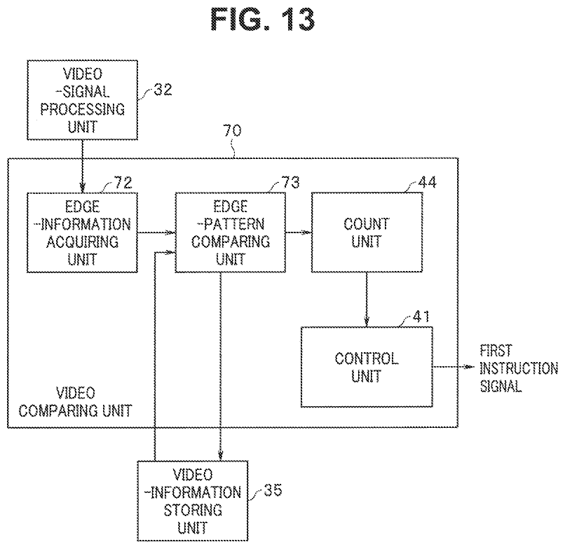

FIG. 13 is a block diagram showing a fifth embodiment of the present invention. An endoscope system in the present embodiment is different from the one shown in FIG. 1 in that a video comparing unit 70 is adopted instead of the video comparing unit 34 in the display apparatus 30. The other components are the same as the components in the first embodiment. Explanation about the components is omitted. FIG. 13 shows an example of a specific configuration of the video comparing unit 70. The video comparing unit 70 is different from the video comparing unit 40 shown in FIG. 4 in that an edge-information acquiring unit 72 and an edge-pattern comparing unit 73 are adopted respectively instead of the color-information acquiring unit 42 and the color-information comparing unit 43. The present embodiment shows an example in which coincidence of shapes of videos is set as a comparison condition.

The edge-information acquiring unit 72 and the edge-pattern comparing unit 73 may be configured by a processor in which a CPU, an FPGA, or the like is used or may operate according to a program stored in a not-shown memory, or a part or all of functions of the edge-information acquiring unit 72 and the edge-pattern comparing unit 73 may be realized by a hardware electronic circuit. The control unit 41 controls the units in the video comparing unit 70.

The edge-information acquiring unit 72 acquires, about an image for display supplied from the video-signal processing unit 32, information concerning a shape of an endoscopic image in an endoscopic image display region. For example, the edge-information acquiring unit 72 uses edge information of the endoscopic image as information concerning an image shape. For example, the edge-information acquiring unit 72 acquires, pixel by pixel, pixel values of all pixels in the endoscopic image display region, detects an edge according to a difference between pixel values of adjacent pixels, and acquires information concerning an edge pattern. The edge-information acquiring unit 72 outputs the acquired edge pattern information to the edge-pattern comparing unit 73.

The edge-pattern comparing unit 73 reads out, from the video-information storing unit 35, edge pattern information stored before a predetermined period and compares the edge pattern information with present edge pattern information inputted from the edge-information acquiring unit 72. When a comparison condition explained below by the edge pattern information is not satisfied, the edge-pattern comparing unit 73 gives the inputted edge pattern information to the video-information storing unit 35 as comparison information and causes the video-information storing unit 35 to update storage. The edge-pattern comparing unit 73 determines whether or not edge patterns of endoscopic image display regions of images for display before and after the predetermined period coincide. When the edge patterns of the endoscopic image display regions coincide, the edge-pattern comparing unit 73 determines that the comparison condition is satisfied. Note that the edge-pattern comparing unit 73 may allow a predetermined error in the comparison of the edge patterns. Even when non-coincidence of edge patterns equal to or smaller than a predetermined number is present, the edge-pattern comparing unit 73 may determine that the comparison condition is satisfied.

The edge-pattern comparing unit 73 performs the comparison of edge pattern information in every predetermined period, for example, every frame, and outputs to the count unit 44 a comparison result about whether or not the comparison condition is satisfied. The count unit 44 outputs to the control unit 41 the number of times the comparison condition is continuously satisfied.

The control unit 41 determines whether or not a count value has reached a predetermined value. The count unit 44 is configured to, when the count value has reached the predetermined value, that is, the edge-pattern comparing unit 73 outputs a comparison result indicating that the comparison condition is continuously satisfied a predetermined number of times, generate a first instruction signal indicating that the mode may be shifted to a burn-in prevention mode and output the first instruction signal to the control unit 31.

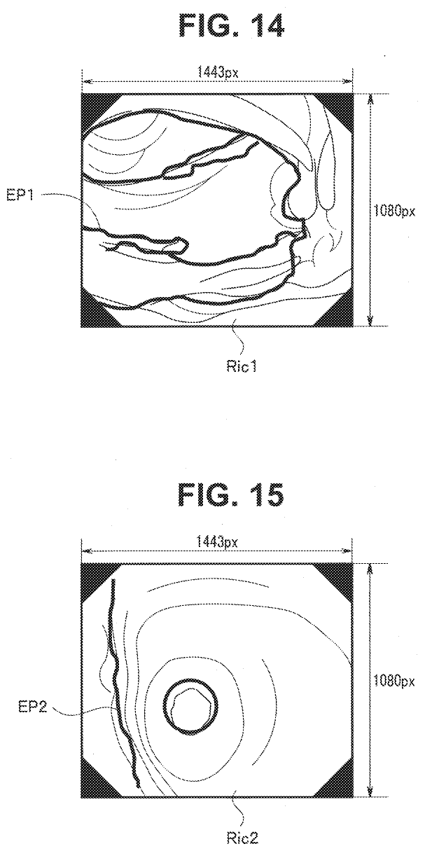

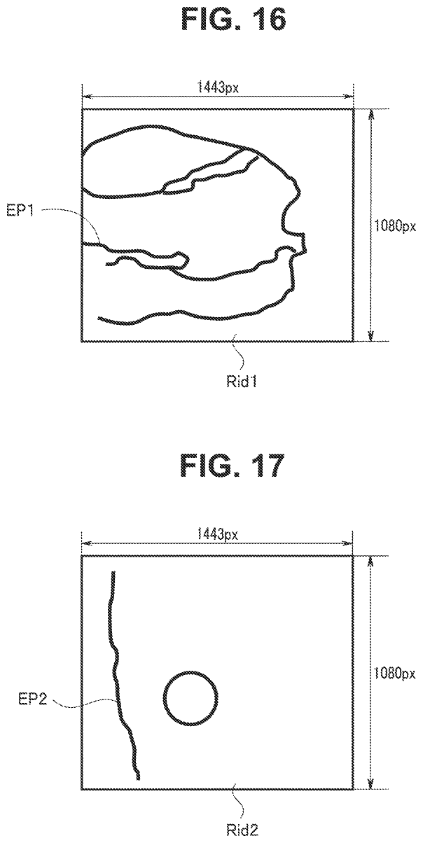

Subsequently, operation in the embodiment configured as explained above is explained with reference to explanatory diagrams of FIG. 14 to FIG. 16. FIG. 14 and FIG. 15 show image portions of endoscopic image display regions of continuously inputted images for display. FIG. 16 and FIG. 17 show edge patterns extracted from endoscopic image display regions respectively corresponding to FIG. 14 and FIG. 15.

It is assumed that images for display Ric1 and Ric2 before and after predetermined one frame are images shown in FIG. 14 and FIG. 15. FIG. 15 shows a present image for display Ric2 after one frame shown in FIG. 14. The edge-information acquiring unit 72 receives an image for display from the video-signal processing unit 32 and acquires edge information in an endoscopic image display region of the inputted image for display. Thick lines in FIG. 14 and FIG. 15 indicate detected edge patterns EP1 and EP2. The edge-information acquiring unit 72 generates an edge pattern from the edge information. FIG. 16 and FIG. 17 respectively show pattern images Rid1 and Rid2 including the edge patterns EP1 and EP2 extracted from FIG. 14 and FIG. 15. Note that a pattern image may be stored or information concerning an edge pattern in the pattern image may be stored in the video-information storing unit 35.

It is assumed that the edge pattern EP1 shown in FIG. 16 based on the endoscopic image Ric1 shown in FIG. 14 is stored in the video-information storing unit 35 at timing when the image for display Ric2 corresponding to FIG. 15 is inputted to the edge-information acquiring unit 72 from the video-signal processing unit 32. The edge-information acquiring unit 72 acquires the edge information shown in FIG. 15, calculates the edge pattern EP2, and outputs the edge pattern EP2 to the edge-pattern comparing unit 73.

The edge-pattern comparing unit 73 compares the edge pattern EP1 shown in FIG. 16 and the edge pattern EP2 shown in FIG. 17 and determines whether or not patterns coincide in the entire endoscopic image display region. In examples shown in FIG. 16 and FIG. 17, it is determined that the patterns do not coincide.

When the patterns coincide in the entire endoscopic image display region, the edge-pattern comparing unit 73 outputs to the count unit 44 a comparison result indicating that the comparison condition is satisfied. The count unit 44 counts the number of times the comparison condition is continuously satisfied and outputs a count value to the control unit 41.

For example, when the count value reaches 18000, the control unit 41 outputs the first instruction signal. For example, when a frame frequency is 60 fps (frames/second), the first instruction signal indicating that the mode may be shifted to the burn-in prevention mode is generated when the comparison condition is continuously satisfied for five minutes.

The other action is the same as the action in the second embodiment shown in FIG. 4.

As explained above, in the present embodiment, the edge information of the endoscopic image display regions is calculated and the edge patterns are acquired, about the images for display before and after the predetermined period, the edge patterns of the endoscopic image display regions coinciding is set as the comparison condition, and the first instruction signal indicating that the mode may be shifted to the burn-in prevention mode is generated when the comparison condition is continuously satisfied for the predetermined period. Consequently, even at a suspension time and the like of an endoscopic test, it is possible to shift to the burn-in prevention mode, and to surely prevent burn-in of the display apparatus.

Note that, in the present embodiment as well, the modification shown in FIG. 3 may be adopted and the burn-in prevention mode may be set based on the first and second instruction signals.

### Sixth Embodiment

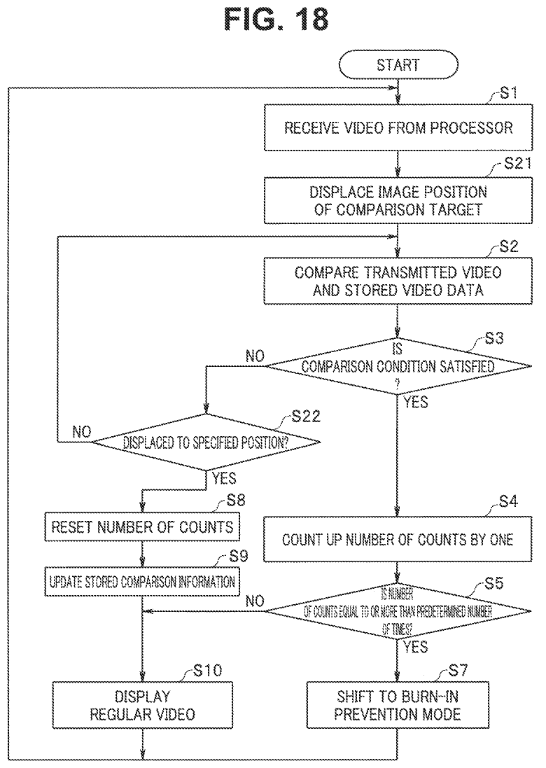

FIG. 18 is a flowchart showing a sixth embodiment of the present invention. In FIG. 18, the same procedures as the procedures shown in FIG. 2 are denoted by the same reference signs and explanation about the procedures is omitted. An endoscope system in the present embodiment is different in determination operations in the video comparing unit 34 shown in FIG. 1, the color-information comparing unit 43 shown in FIG. 4, and the edge-pattern comparing unit 73 shown in FIG. 13. The other components are the same as the components shown in FIG. 1. Explanation about the components is omitted. FIG. 18 shows the determination operations in the video comparing unit 34 shown in FIG. 1, the color-information comparing unit 43 shown in FIG. 4, and the edge-pattern comparing unit 73 shown in FIG. 13. The present embodiment shows an example in which a comparison target is displaced by a predetermined number of pixels in at least one direction of horizontal and vertical directions and it is determined whether or not a comparison condition is satisfied.

FIG. 18 is different from FIG. 2 in that step S21 is provided between steps S1 and S2 and step S22 is provided between step S3 and step S8. In step S21, processing for displacing an image position is performed about a comparison target. In the respective embodiments explained above, in the case in which whether or not the comparison condition is satisfied is determined according to the comparison before and after the predetermined period, the comparison is performed using the information concerning the same position in the endoscopic image display regions of the two images. However, even at a suspension time and the like of an endoscopic test, it is conceivable that the endoscope scope 10 is moved by wind or the like and an image for display moves in a screen. In this case, an endoscopic image displayed in an endoscopic image display region is an endoscopic image, an image position of which is shifted as a whole. Therefore, even in such a case, in order to satisfy the comparison condition, one of two comparison target images or one of pieces of information concerning the two images is shifted in pixel units, and determination is performed.

Step S21 is a step of performing processing for displacing, for example, by one pixel, one of two comparison target images or one of pieces of information concerning the two images. In step S2, comparison is made in a state in which inputted information concerning an image for display or the comparison information read out from the video-information storing unit 35 has been displaced by predetermined pixels in step S21.

In this state, it is determined in step S3 whether or not the comparison condition is satisfied. When the comparison condition is satisfied, the processing is proceeded to step S4 and the number of counts the comparison condition is continuously satisfied is counted up. When the comparison condition is not satisfied, the processing is shifted to step S22. It is determined whether or not the position displacement in step S21 proceeds to a specified position set in advance.

The position displacement in step S21 is performed by a predetermined number of pixels at least in one direction of horizontal and vertical directions. When the comparison is performed about all pixels positions for which the position displacement is permitted, the processing shifts to step S8 and the number of counts is reset. When the comparison is not performed for all the pixel positions for which the position displacement is permitted, the processing is returned to step S21 and processing for displacing one of two comparison target images or one of pieces of information concerning the two images by, for example, one pixel is performed.

In this way, the position is displaced in a predetermined range and it is determined whether or not the comparison condition is satisfied. Consequently, even when an image is displaced by several pixels by, for example, wind, it is possible to surely generate the first instruction signal indicating that the mode may be shifted to the burn-in prevention mode.

The other components and action are the same as the components and action in the respective embodiments explained above.

Note that, in the present embodiment as well, the modification shown in FIG. 3 may be adopted and the burn-in prevention mode may be set based on the first and second instruction signals.

As explained above, in the present embodiment, at a suspension time and the like of an endoscopic test, it is possible to surely shift to the burn-in prevention mode, even when an endoscope is moved by wind or the like.

Note that the second to sixth embodiments explained above can be used in combination with one another.

For example, first, the third or fourth embodiment in which the luminance information is used may be adopted and, subsequently, the second embodiment in which the color information is used and the fifth embodiment in which the edge pattern is used may be adopted. In this case, in the determination using the luminance information, the mode may be shifted to the burn-in prevention mode when the number of counts is 1. It is possible to immediately set the burn-in prevention mode by turning off the light source apparatus. Subsequently, the shift to the burn-in prevention mode is determined by the comparison of the colors or the comparison of the patterns. Consequently, even when the light source apparatus is not turned off, it is possible to surely shift to the burn-in prevention mode.

In the respective embodiments explained above, an example is explained in which the video comparing unit, the video storing unit, the operation detecting unit, and the control unit are configured in the display apparatus. However, these components may be provided in a video processor. A display image corresponding to the burn-in prevention mode may be outputted to the display apparatus by the video processor.

Note that, among the techniques explained in the specification, the control explained mainly in the flowcharts can often be set by a program and is sometimes stored in a recording medium or a recording unit. As a method of recording in the recording medium or the recording unit, the control may be recorded at a product shipment time, may be recorded using a distributed recording medium, or may be downloaded via the Internet.

The execution order of the steps in the flowcharts may be changed, a plurality of the steps may be simultaneously executed, or the steps may be executed in different order in every execution unless contrary to natures of the steps.

Note that, in the embodiments, the portion described as “unit” may be configured by a dedicated circuit or may be configured by combining a plurality of general-purpose circuits or may be configured by combining, according to necessity, processors such as a microcomputer and a CPU that perform operation according to software programmed in advance or sequencers such as an FPGA.

The present invention is not limited to the respective embodiments explained above per se. In an implementation stage, the constituent elements can be modified and embodied in a range not departing from the gist of the present invention. Various inventions can be formed by appropriate combinations of a plurality of constituent elements disclosed in the respective embodiments explained above. For example, several constituent elements among all the constituent elements explained in the embodiments may be deleted. Further, the constituent elements in different embodiments may be combined as appropriate.

What is claimed is:

1. A display system comprising:
a memory configured to store comparison information; and
a processor, wherein
the processor receives an image for display including an endoscopic image obtained by picking up an image with an endoscope, generates a display image used for display, outputs the display image to a display, compares information concerning the endoscopic image included in the inputted image for display and the comparison information stored in the memory, obtains a comparison result, and controls, according to the comparison result, whether or not to operate in a burn-in prevention mode.

2. The display system according to claim 1, further comprising a display configured to display the display image, wherein
the memory stores information concerning an image for display before a predetermined period inputted to the processor as the comparison information.

3. The display system according to claim 2, wherein, when a comparison result indicating that a predetermined condition is not satisfied in the comparison of the information concerning the endoscopic image and the comparison information is outputted by the processor, the memory updates the information concerning the image for display.

4. The display system according to claim 2, wherein the processor compares color information of the endoscopic image in the inputted image for display and color information of an endoscopic image in an image for display stored in the memory.

5. The display system according to claim 2, wherein the processor compares luminance information of the endoscopic image in the inputted image for display and luminance information of an endoscopic image in an image for display stored in the memory.

6. The display system according to claim 2, wherein the processor compares an edge pattern of the endoscopic image in the inputted image for display and an edge pattern of an endoscopic image in an image for display stored in the memory.

7. The display system according to claim 2, wherein
the memory stores an average luminance value of the endoscopic image as the comparison information, and
the processor compares an average luminance value of the endoscopic image in the inputted image for display and the average luminance value stored in the memory.

8. The display system according to claim 1, wherein, when the information concerning the endoscopic image included in the image for display and the comparison information stored in the memory satisfy a predetermined comparison condition, the processor obtains a comparison result for shifting to the burn-in prevention mode.

9. The display system according to claim 1, wherein, when the information concerning the endoscopic image included in the image for display and the comparison information stored in the memory continuously coincide a predetermined number of times, the processor regards that a predetermined condition is satisfied and obtains a comparison result for shifting to the burn-in prevention mode.

10. The display system according to claim 1, wherein the processor detects a non-operation state for the display, obtains a detection result, and controls, based on the comparison result and the detection result, whether or not to operate processing in the burn-in prevention mode.

11. The display system according to claim 2, wherein the processor obtains a comparison result based on at least one comparison among comparison of color information of the endoscopic image in the inputted image for display and color information of an endoscopic image in an image for display stored in the memory, comparison of luminance information of the endoscopic image in the inputted image for display and luminance information of the endoscopic image in the image for display stored in the memory, comparison of an edge pattern of the endoscopic image in the inputted image for display and an edge pattern of the endoscopic image in the image for display stored in the memory, and comparison of an average luminance value of the endoscopic image in the inputted image for display and an average luminance value stored in the memory.

12. An endoscope system comprising a display system including a display, a memory configured to store comparison information, and
a processor, wherein
the processor receives an image for display including an endoscopic image obtained by picking up an image with an endoscope, generates a display image used for display, outputs the display image to a display, compares information concerning the endoscopic image included in the inputted image for display and the comparison information stored in the memory, obtains a comparison result, and controls, according to the comparison result, whether or not to operate in a burn-in prevention mode.

13. A display control method comprising:
receiving an image for display including an endoscopic image obtained by picking up an image with an endoscope and generating a display image used for display;
comparing information concerning the endoscopic image included in the inputted image for display and comparison information by using information concerning the image for display before a predetermined period as the comparison information, and outputting a comparison result; and
updating the information concerning the image for display when the comparison result indicating that a predetermined condition is not satisfied in the comparison of the information concerning the endoscopic image and the comparison information is outputted and, when the information concerning the endoscopic image and the comparison information continuously coincide a predetermined number of times, regarding that the predetermined condition is satisfied and shifting to a burn-in prevention mode.
